(19) United States
(12) Patent Application Publication
(10) Pub. No.: US 2009/0088611 A1
(43) Pub. Date: Apr. 2, 2009

(54) METHODS AND DEVICES FOR CONTINUOUS AND MOBILE MEASUREMENT OF VARIOUS BIO-PARAMETERS IN THE EXTERNAL AUDITORY CANAL

(76) Inventor: Johannes Buschmann, Muenchen (DE)

Correspondence Address: PATENT CENTRAL LLC;Stephan A. Pendorf, 1401 Hollywood Boulevard, Hollywood, FL, 33020, US

(21) Appl. No.: 12/239,411

(22) Filed: Sep. 26, 2008

(30) Foreign Application Priority Data
Sep. 27, 2007 (DE) 10 2007 046 295.8

(51) Int. Cl. A61B 5/00

(52) U.S. Cl. 600/301

(57) ABSTRACT

A method and a device for the continuous, mobile non-invasive and aesthetically unobtrusive measurement of important vital parameters, in particular body(core)temperature, arterial oxygen saturation, heart rate, respiration rate (by pulse oximetry), blood pressure, ECG, and substance concentrations in blood or tissue. The measuring site and the position of the sensor components is the (proximal) auditory canal, whereby there results an unobtrusive sensor technology suitable for a stable monitoring in everyday life i.e. even unaffected by motion. The sensor system includes a small evaluation unit and electronics for signal processing and e.g. means for wireless transmission to a mobile phone. Thus physiological parameters become available for long term diagnostics, for outpatients, for monitoring during rehabilitation, or for monitoring the health status in everyday life, while doing sports and during training, for an increase of the safety of individuals or of people with dangerous occupations or risky hobbies.

### FIELD OF THE INVENTION

The invention relates to a method as well as to a device for non-invasive measurement of important physiological parameters in humans or other animals in particular continuously and under mobile conditions. To these parameters belong in particular the measurement of the body(core)temperature, tissue-optical parameters like the arterial oxygen saturation, heart rate and respiration rate, furthermore the concentrations of certain substances in blood and/or tissue without taking samples of blood fluids, but also the measurement of blood pressure, ECG, as well as mechanical parameters such as position, location and acceleration. As a measuring site the auditory canal is used.

### BACKGROUND OF THE INVENTION

Modern medicine shows a trend towards less invasive interventions (minimally invasive surgery) and towards shorter and shorter stays in hospital. Today many diagnostic and therapeutic measures, many surgical interventions which were earlier carried out in a hospital are outpatient care today or at least performed in clearly shorter hospitalization time. This trend should logically be flanked by monitoring technologies to collect information about the outpatient's recovery progress i.e. to monitor potential health risks more or less continuously and to extend the hospital's professional service up to wherever the patients chose to stay. Many risks are of continuous nature and, hence, accompany the everyday life as for example diabetes, allergies, hypertension, chronic obstructive pulmonary disease (COPD), fat metabolism disorders etc.

Not only patients, also healthy, mobile individuals become increasingly aware of the value of health and try to remain in good condition and even to improve their health status by various activities such as training, visiting fitness studios, leisure activities, reasonable dietetics etc. On the other hand they also like to be informed, whether important (prognostic) parameters are all right, in particular whether the health-supporting measures actually prove effective or e.g. whether promised training effects can be objectified and the like.

Finally some occupational groups, e.g. pilots, fire-fighters, divers, cold-storekeepers, mountain rescuers, blast furnace workers, glassblowers, etc. are potentially endangered by oxygen depletion, respirable dust, by hypo- or hyperthermia. These occupational groups wish to quantify this danger by monitoring relevant physiological parameters in order to estimate the danger or to be able to restrict it.

The development of medical sensor technology until today permits the access to diagnostically important bio-parameters such as lung function, end-tidal pCO₂, blood pressure, body temperature, oxygen saturation, electrocardiogram (ECG) and so on. Apart from very few exceptions (12-channel long-term ECG or 24-hours-blood pressure measurement) most physiological parameters are restricted to one single measurement or short term measuring courses only, or they are accessible under clinical conditions (=intensive care monitoring) only. Many biochemical parameters even need taking blood tests.

### Measurement of the Body(Core) Temperature

There are a number of possibilities for the measurement of body temperature which have in common, however, that mostly only the maximum temperature is measured and that only one single measurement is taken. The body temperature is monitored as a continuously measured variable only under the conditions of intensive care.

The measurement of the body temperature can be done in principle at different more or less suitable places, the measured temperature being more or less relevant for what is called “body(core)temperature” accordingly. Body(core)temperature on the one hand means a central temperature in a spatial-anatomical sense, a temperature of the most internal organs which is not disturbed by immediate external influences.

Body(core)temperature on the other hand means a temperature measured close to the hypothalamus since here the physiological sensor for the temperature control is located and thus a temperature measured here has the most importance and is most meaningful for the thermal balance of the organism. From this point of view a body(core)temperature measured in the external auditory canal is exceedingly relevant.

In the auditory canal only radiation sensors are described, under the idea that the radiation from the rear auditory canal represents the body(core)temperature. However, radiation sensors are not very precise. Up to now measurements of the body temperature in the auditory canal are carried out discontinuously i.e. as single shot measurements or as one out of a few within a short period.

The body(core)temperature is an important physiological value for general monitoring, because it reflects many important conditions of the body. The value of the parameter “continuously-in-everyday-life-measurable body(core)-temperature”, its practical availability and, above all, the physiological information which can be derived from this parameter can not even be predicted today. It has to be seen which information can be gained with the help of this measured variable, as it turned out what meaning a long-term ECG or a 24 hours of blood pressure measurement has. In the following interesting applications for a continuous measurement of the body(core)temperature are outlined:

The body(core)temperature increases with physical activity, so that the body(core)temperature can be used in the professional field as well as in the sports to tune physical activities. It is absolutely conceivable that in the close future sportsmen will be exchanged with the help of their body(core)temperature, e.g. if they have reached a critical temperature—based on the idea that hyperthermia indicates an unphysiological condition which is accompanied by a decrease of physical capability.

The body(core)temperature is also related to the basal metabolism which again is influenced by the thyroid hormones, but also by adrenaline and by the growth hormone as well as by other neurotransmitters.

The body(core)temperature is influenced by gestagens which cause an increase of the target temperature, so that the second half of the woman's cycle can be detected by an increase of the basal body temperature. Thus the body(core)temperature is a value which allows to identify the ovulation and the so-called “fertile days of the woman” as well.

The body(core)temperature is a controlled value. Accordingly the actual value usually corresponds to the target value. However, the target value can be shifted, for example, by pyrogens, so that fever appears, in the sense of a poisoning by bacterial toxins, for example, during infectious diseases. But allergic reactions can be accompanied by fever, too, e.g., repulsion reactions of grafts, transfusion reactions, vaccination reactions, snakebite and other inoculations of animal poisons or antigens of any kind.

The temperature control is anaesthetized by many substances which sedate the central nervous system, e.g., alcohol or hypnotics in higher doses and narcotics. Then the body(core)temperature approaches ambient temperature, the faster, the lower the caloric resistors are. In principle the body(core)temperature can deviate in both directions, a threatening situation which needs to be diagnosed to be able to treat it.

Furthermore the body(core)temperature is closely related to the vigilance. Regardless of purely physical activity the body(core)temperature changes as a function of the vigilance. Thus the body(core)temperature can be used for the monitoring of the vigilance.

Also the sleep-wake rhythm changes the temperature control: In the REM phases of the sleep the temperature control is reduced, while it is normal in the other sleep phases.

The detection of states of fatigue is of crucial importance in particular the detection or better still the prediction of microsleep events while driving. It is subject to further investigations, as to which extent the body(core)temperature or another of the proposed bio-parameters is suitable as an indicator for this.

The U.S. Pat. No. 6,694,180 teaches an attachment temperature sensor in the auditory canal however with a different way of attachment to the auditory canal: our embodiments comprise a sensor-carrier which creates the attachment force by having opposing points or an opposing distribution of points, at least two. In the U.S. Pat. No. 6,694,180 no sensor-carrier according to our specifications is described as an element of attaching the probe to the wall of the auditory canal.

### Measurement of the Blood Pressure:

The measurement without taking blood samples, i.e. the non-invasive measurement of blood pressure is up to now exclusively based on the use of cuffs, usually around an arm, seldom around a finger. These blood pressure cuffs have a constant outer circumference and permit the centripetal compression of the tissue up to the complete occlusion of the arteries and arterioles crossing the tissue. Other measuring sites which would permit a mobile, non-invasive blood pressure measurement are not described.

U.S. Pat. No. 4,029,083 describes an audiometric device for analyzing hearing making use of an inflatable bladder which can be stuck into the auditory canal. The device comprises several tubes reaching from outside into the auditory canal for various analytic purposes. The application describes an inflatable bladder reaching into but not fully within the auditory canal for producing changes of the pressure within the auditory canal but not for measuring the blood pressure which would not be possible this way.

The WO27100958 A1 shows a plurality of sensors and a plurality of parameters not teaching how any of the sensors might measure anything. An exception to this rule is the explanation of an inflatable balloon for measuring the blood pressure by auscultation—a technology we do not claim since auscultation is highly erroneous in an organ into which sound is lead by the funnel characteristics of the auricle and which is meant to deal with sound. The crucial difference to our approach is that the WO27100958 A1 analyzes in combination with stimulation which we do not—this invention deals purely with measurements of parameters without any stimulation.

### ECG:

The classical sites for placing ECG electrodes are on the thoracic wall. Other places are not in use in the adult ECG measurement. In the fetus an ECG is derived from the scalp, indeed, with the motherly body/abdomen as a counter-electrode or as an authoritative electrode. A mobile ECG without sticking of electrodes or without clothes containing electrodes is not available up to now. The U.S. Pat. No. 4,601,794 describes a non-invasive, external ear canal electrode useful for transmitting sound stimulus to an ear canal for conducting electrical signals picked up from the ear canal epidermal surface as a result of the stimulus. We have an apparatus for diagnosing hearing defects here making use of electrodes in the auditory canal. ECG diagnostics are neither intended nor claimed.

Also US 20070112277A1 has a bioelectric analytic at heart making use of electrodes in the auditory canal, too. Again an acoustic stimulus is used and electric potentials analyzed to reach an EEG. Again an Electrocardiogram with the use of intra auditory canal electrodes is neither intended nor claimed.

### Measurement of Photometrical and Optic-Plethysmographic Values Pulse Oximetry:

The pulse oximetry is an optical process to monitor the oxygen saturation by means of photometry of the tissue containing both pulsating species of the arterial hemoglobin-dyes, Hbₒₓ and Hb_{red}. It is widely used and highly miniaturised already. A mobile pulse oximetry fails nowadays because of a missing suitable measuring site: the measuring site is almost always the finger; other measuring sites are less reliable. Thus a mobile pulse oximetry in the everyday life is not available up to now, although there is a high need for it.

An access to the information oxygen depletion is important in two ways: Firstly the functionality and integrity of the brain is quickly and dangerously threatened by oxygen depletion. Secondly it would be the mobile and continuous measurement of the oxygen saturation which allows a diagnostic look at a disease of modern civilization becoming more and more frequent and with a high number of unrecorded cases and often a long period between disease beginning and diagnosing: the COPD, the chronically obstructive lung disease. Smoking, respirable dust in the air or allergies disturb the expiration and lead fairly long-term to COPD, which is responsible today already for approx. one third of all deaths. The parameter oxygen saturation allows the detection of diminished lung function in particular under strain. The use of a sensor bundle oxygen saturation, heart rate and respiration rate, the diagnosis COPD may be already put under circumstances at a time in which the prognosis is still clearly better.

Besides, the parameter oxygen saturation is then an indicator of a danger, when decreased oxygen partial pressures are expected or, nevertheless, at least possible. Occupational groups like firefighters, pilots etc., however, also activities like mountaineering, diving etc. profit from this sensor system.

An important element of the pulse oximetry is that the parameter oxygen saturation is global, i.e. everywhere in the body predominates the same arterial oxygen saturation. This is due to the fact that the arteries and arterioles merely distribute the blood; a diffusion of oxygen does not occur. Therefore the arterial oxygen saturation can be measured by means of the pulse oximetry at every place and the measured arterial oxygen saturation is valid for any other place of the body.

The auditory canal as a measuring site for the pulse oximetry was considered only a little and also only under rather special application scenarios like fighter pilots and similar—see the U.S. Pat. No. 5,213,099 and the U.S. Pat. No. 5,662,104.

The U.S. Pat. No. 5,213,099 suggests an elastic ear plug which contains the optical components for the pulse oximetry. The mechanical arrangement is simple, methods of the suppression of shunt light are not described. Considerations for the optimizations of the auxiliary variable omega or to the limitation of motion artifacts are not contained. A reflective light path through the tissue is discussed, which shows an unfavorable light path, because the reflex pulse oximetry contains considerable sources of error. The patent has run out after the 8^{th} year. It shows an initial stage of ear pulse oximetry, which was obviously not pursued further.

The U.S. Pat. No. 5,662,104 suggests a grip in witch one optical component comes to lie in the most external area of the auditory canal, the other optical component is completely outside. The grip with both optical components is wide-open and promises little hold. Shunt light seems unlikely. The optical path extends between outside and inside, i.e. in the most external edge of the auditory canal's entrance and not in the auditory canal itself. The transilluminated tissue is an extremely flat (dull-angular) wedge—the smallest changes of the grip's position as they are inevitable with motion, cause huge changes of the tissue thickness and thus signals which make no sense.

The WO 05020806 also intends to measure oxygen saturation from within the auditory canal. In contrast to this invention not the circumference not the auditory canal is transilluminated but a “distal bend” of the auditory canal is transilluminated. It is not even clear whether actually “pulse oximetry” is meant since just “oximetry” is mentioned and the word saturation is never linked with the arterial oxygen saturation, an important difference in the technologies used. Several substantial disadvantages are associated. There is a first and a second position to be reached by the optical components in order to transilluminate a certain bend of the auditory canal and it remains unclear how the user will accomplish to position the device so that the exact positions for the transillumination are reached. The light path is a crucial element of pulse oximetry, if the light path involves shunt light, the oxygen saturation values calculated become meaningless. An important disadvantage of the WO05020806 is, that the positioning of the optical components “proximal to the tympanon”, a very sensitive region close to the tympanic membrane becomes extremely unpleasant and any potential attachment of an optical component to the wall of the auditory canal would even be painful (comparable to the cornea of the eye) but is a necessary prerequisite of pulse oximetry if the application even refers to this method). The WO05020806 teaches no shunt light considerations, no light path considerations done. No considerations of how motion stability can be reached in order to avoid motion artifacts, again an important problem.

The U.S. Pat. No. 6,080,110 is the only patent which deals with mobile monitoring in combination with the auditory canal as the site of measurements. The one and only parameter dealt here is the heart beat. It is accomplished by making use of a optical tissue sensor in the auditory canal using one wavelength only so no oximetry and no pulse oximetry is possible. For detecting the heart beat a reflectance method is used. WE teach a transillumination method referred to as transmission pulse oximetry a significant difference. To be even more specific: we teach a “circummission pulse oximetry” an entirely new subspecies of the transmission pulse oximetry.

The U.S. Pat. No. 6,694,180 further teaches a SpO₂ sensor, but it is outside the auditory canal.

The WO5020841 (U.S. Pat. No. 7,107,088) is limited to animal monitoring. It shows a plurality of sensors and a plurality of parameters not teaching how any of the sensors positioned in the auditory canal might measure anything, it does not teach what to take care of, what obstacles to avoid. Specifically the application does not teach our sensor mechanics i.e. the sensor-carrier and the sensor-carrier-positioning-element nor any technologies like our light paths for SpO₂ or the creation of the attachment forces for a temperature sensor and the like.

The WO027100959 A2 and A3 teaches sensors in combination with either a neck collar or a control circuit comprising a blinking control light which emits light in accordance with the sensed vital signs. None of this relates to our teachings, i.e. the disclosed material is quite far away.

All patent applications together show rather clearly how difficult it is to fulfill even the most important requirements of the pulse oximetry at the same time. Thus the U.S. Pat. No. 5,662,104 exemplarily solves the shunt light problems, however, has problems with the hold of the sensor and with light ways susceptible to motion. On the other hand, the U.S. Pat. No. 5,662,104 has stable ratios of the tissue layer thickness, however, does not care about the error source shunt light. On top of that, the sensor is mechanically unsteady, the attachment is based exclusively on the expansion of the material. There is no mechanical connection with the auricle.

Measurement of Mechanical Values like Position, Acceleration and Location:

Mechanical sensors are known for a long time, sufficiently small sensors, however, became only recently available which allow construction and design of unobtrusive mobile sensor systems, e.g. 3 axes acceleration sensor ADXL330 of just 4×4×1.45 mm. Not available until now in the field of mobile sensor technology are mechanical values let alone the combination of mobile sensors with other parameters witch are a also part of the invention disclosed here.

### BRIEF SUMMARY OF THE INVENTION

A prerequisite for monitoring bio-parameters of mobile people, i.e. under everyday life conditions is that sensors are available which meet three important requirements: firstly, they should perform under the condition of physical mobility, i.e. in a person who lives a normal, unspectacular everyday life, e.g., at work or leisure time. Secondly, they should be cosmetically unobtrusive, not irritate in everyday life, neither physically nor socially, i.e. neither the wearer nor his environment. Thirdly, the sensors should measure continuously, often, or at least repeatedly. In respect to this, “continuously” means that the frequency of the measurements is so high that changes of a measured variable are determined (reading of samples) with a frequency which is sufficient for all diagnostic and/or therapeutic purposes that a quasi-continuous information can be obtained. An important problem in medical data collection is an insufficient sample rate, a measurement error source, not seldom seen, in particular if signal courses exceed typical over a time period longer than should be looked at, e.g., about a day's course. Sensors which meet these requirements are very demanding and innovative in comparison to sensors for discreet, single measurement values.

The following demands must be met by a modern, mobile, continuous sensor system applicable to every day life:
the sensors should be mobile i.e. concerning the choice of the measuring site and the measuring principle designed in such a way that they continuously provide data and are very resistant towards irritations which arise from the mobility of the person to be monitored.
mobile sensors should be cosmetically acceptable, i.e. they should be very small and hard to be seen and disturb everyday life as little as possible.
mobile sensors should be designed in respect to their power consumption in such a way that the available energy cells provide an acceptable operation time.
sensors should be applied in suitable measuring sites. Thus, measuring sites like mouth, esophagus, rectum or vagina are not reasonable, measuring sites like finger, nose forehead etc. are not practicable.
mobile sensors should be put on the body in such a way that they take up primarily as little as possible disorders under normal terms, i.e. during the designated operation, e.g., in the everyday life; disorders based on motions (motion artifacts) like shock and other mechanical irritations, or disorders on the basis of external influence like illumination, irritations by water, thermal, optical or electromagnetic influence.

The invention refers to methods and devices for measuring at least one physiological and/or biochemical parameter in the auditory canal of a human or an animal and at least one sensor-carrier which is positioned in the auditory canal, at least one sensor component, that is positioned on the sensor-carrier and/or is connected with it, for the measurement of at least one physiological or biochemical parameter respectively; furthermore a sensor-carrier-positioning-element which positions the device in the auditory canal and which is connected at one end with the sensor-carrier, that sensor-carrier-positioning-element defining the penetration depth of the sensor-carrier in the auditory canal and being held by the sensor-carrier in the auditory canal.

According to the invention the external auditory canal is used for the described innovative sensor system as the measuring site which is especially well suited for the mobile and the continuous sensor system respectively due to the combination of physiological properties on the one hand with mechanical, technical and functional ones according to the invention, on the other hand.

### BRIEF DESCRIPTION OF THE DRAWINGS

Further details, advantages and features of the invention can be gathered from the following description of an exemplary embodiment on the basis of the drawing, in which:

FIG. 1 illustrates the sensor device;

FIG. 2 illustrates the sensor-carrier;

FIG. 3 is a cross-section through the external auditory canal with sensor component on an single sensor-carrier;

FIG. 4 is a cross-section (longitudinal) through the external auditory canal with sensor component on double sensor-carrier;

FIG. 5 illustrates the light ways in the cross section through the external auditory canal;

FIG. 6 illustrates net light path and shunt light with photo-metrical or optical plethysmographic sensor technology;

FIG. 7 illustrates diagonal light ways and spiral type light ways with a double sensor-carrier with 180° positioning of the sensor components;

FIG. 8 illustrates the meander-like temperature sensor to the measurement of the body(core)temperature or meander-like (ECG) electrode;

FIG. 9 illustrates the expansionary collar for the monitoring of the blood pressure;

FIG. 10 illustrates the functional connection of the system components;

FIG. 11 illustrates an alternative embodiment of a sensor-carrier with 2 self-positioning contact points;

FIG. 12 illustrates a sensor device with alternative embodiment of a sensor-carrier with 3 self-positioning contact points; and

FIG. 13 represents a principle embodiment of a pressure sensor for detecting of the blood pressure in the external auditory canal.

### DETAILED DESCRIPTION OF THE INVENTION

### 1. Definitions

For the purposes of the present patent application the following definitions are used as a basis:

“Continuous monitoring of a parameter” means that either measurement values are available on a truly continuous basis, or that discreet, separate measurement values can be gained discontinuously at a temporal sample rate which is high relative to the biological kinetics of the parameter and which samples are sufficient for the intended diagnostic or therapeutic purpose. Thus the necessary sample rate for the measurement value depends on the temporal kinetics with which the parameter to be measured is changing.

“Sensor design” primarily means the sensor design in respect to its functionality in particular concerning the signal quality—a design as to aesthetic points of view is a secondary aspect.

“Light” means electromagnetic radiation in the range of 400 nm to 2500 nm, in particular in the range between 600 nm and in 1100 nm.

“Sensor component” means the sensor or probe in the essential sense, that is to say the device, which touches the biological structure or the component or which converts the essential physiological or biological value into an electric signal, for example, converters, semiconductors, sensors, electrodes, tension measurement probes and so on.

“Sensor-carrier” and “sensor-carrier-positioning-element” [in former applications also referred to as “sensor-carrier-form-tube”] mean functionally and mechanically actively-linked devices which jointly position the sensor component relative to the auditory canal in such a way that the physiological or biological parameter can be measured as noise-free as possible.

From a formally logical point of view sensor-carrier and sensor-carrier-positioning-element have distinct separable tasks: it is the task of the sensor-carrier to define the radial position of the sensor component and its attachment force inside the auditory canal; it is the task of the sensor-carrier-positioning-element to define the axial position of the sensor component relative to the auricle and at the same time of the sensor-carrier, to which it is mechanically engaged. When it comes to concrete embodiments the sensor-carrier and the sensor-carrier-positioning-element can be either distinguishable elements or both elements indistinguishably melt together. The functions however remain separate as mentioned above.

“Sensor or sensor device” means the combination of sensor component, sensor-carrier, sensor-carrier-positioning-element, miniature processing unit and other devices which form altogether the entire sensor. It is the combined effectiveness of some or all of these components with important principles of the sensor design and further methods according to the invention which result in the intended functionality of the sensor.

“Absorption” actually relates to two different optical phenomena: on the one hand absorption means light intensity which can be measured after the passage through the tissue at the light detector, regardless of whether the input light intensity or the electric power is known; on the other hand, it means the ratio of the light intensity radiated into the tissue or its substructures relative to the light intensity after the passage through the tissue or its substructures.

“Shunt light” refers to two phenomena of the light propagation within the tissue, in particular of pulse oximetry:

“Shunt light” means that light travels from the light emitter to the light sensor on a direct path.

“Shunt light” in a narrow sense means that light from the light emitter reaches the light sensor on a direct path without having transilluminated through tissue perfused by blood on its way. If tissue is transilluminated which is not perfused by blood in particular through which no arterioles are passing, it can be dealt with as shunt light in a narrow sense. Dealing with reflex-pulse-oximetry shunt light in the narrow sense can not be avoided practically, because the light also transilluminates tissue which is not perfused by blood, e.g. callused skin or external skin layers. If the light is reflected at the tissue surface, e.g. the skin surface, a part of the light reaches the light sensor. This shunt light has not transilluminated the tissue. Dealing with transmission-pulse-oximetry practically no shunt light in the narrow sense reaches the sensor that has transilluminated tissue not being perfused by blood.

Shuntlight must be dealt with the construction of an optical sensor, i.e. with the sensor design in the best possible way.

“Net-light-path” means the shortest light path which the light can use from the point of leaving the light emitter up to the point of entering into the light sensor—including scatter and diffraction phenomena in the vital tissue. On the way from the light emitter to the light sensor any number of propagation ways of various lengths is conceivable, is possible or realized respectively, depending on the multiple places/scatter-centers at which the light could be bent, refracted and scattered. The “net-light-path”, however, represents the special light path with the highest light intensity at the light sensor—including scatter and diffraction phenomena in the vital tissue.

“Mechanical parameters” mean values like position, location, acceleration and quantities derived from those e.g. by integration or differentiation.

“Circummission Pulse Oximetry” and “Circummission Light Path” relate to the very special light path through the wall of the auditory canal. The circummission light path is by no means correctly characterized by the “reflection pulse oximetry” light paths since this term implies that the optical components are next to each other—meaning at a distance of a few millimeters on the same side of the skin i.e. a more or less flat piece of tissue. The tissue of the auditory canal can be considered flat only in a radial sense i.e. if the optical components were spaced in the direction of the auditory canal's longitudinal extension (depth). “Transmission pulse oximetry” is also not an ideal term, since it refers to a net light path which extends between opposing points of a tissue layer which is transmitted by light straight through such as a finger or an ear lobe or a subdermal piece of tissue in a fetus. The net light path through the auditory canal, the circummission light path, belongs to none of the classical pulse oximetry species: here the straight light path is not only not used, it is deliberately blocked and the net light path is constantly bent, in a half circle type, either direction being equally valid. Accordingly the terms “Circummission Light Path” and “Circummission Pulse Oximetry” seam most appropriate for measuring light absorption in the auditory canal i.e. for a tissue optical principle in which both, tissue and net light path are bent. Optical components are positioned at 180° opposing places—relative to a cross section through the tube and thus generate a half circle net light path; if there is an axial shift of the two optical components, the net light path turns into a half-spiral—again equally valid in either direction.

The auxiliary variable Ω, the ratio of the modulation depths contains the oxygen information (oxygen saturation of the pulsating blood). But Omega contains other factors too: It also depends on the optical spectra emitted by the light emitters, which are mostly LEDs and by the spectral sensitivity of the receiver, mostly a photodiode. It also depends on the hemoglobin absorption spectra of the two species involved in pulse oximetry, oxihemoglobin and desoxihemoglobin. Combining theses influences, omega depends on the two spectral transfer function from emitters to the receiver. There is one more factor omega depends on, which is unknown or unobserved: the shunt light. The lower the shunt light fraction the smaller is omega at a given pair of spectral functions and at a given oxygen saturation e-g—100%. Thus omega becomes a quality factor, which can be influenced by the sensor design.

The most important means to optimize omega i.e. to get it as low as possible at a given oxygen saturation are the avoidance of shuntlight and the optimization i.e. maximization of both modulation depths separately. The optimization of omega is a complex issue concerning the light path.

Means to minimize the shunt light for an optic transmission sensor in the auditory canal are two: blocking the optical coupling between emitter and receiver and blocking even the optical coupling between the tissue areas surrounding emitter and receiver as effectively as possible.

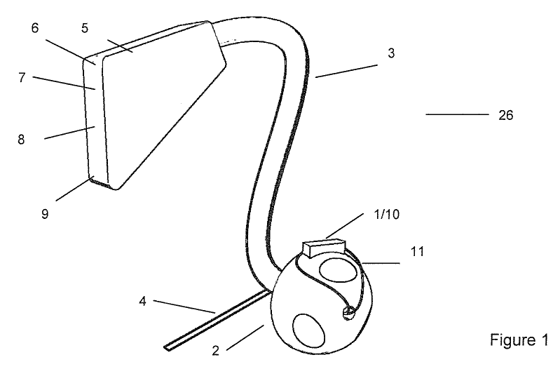

FIG. 1 shows the sensor device (26) with its most important components. The miniature evaluation unit behind the auricle (5) is connected with the sensor-carrier (2) via the anatomically adapted, preformed sensor-carrier positioning element (3). The fixation thread (4) additionally fixates the sensor device (26) in the auricle. The sensor component (1), e.g., a temperature sensor component (1) is mounted on the sensor-carrier (2) and connected with the connection wires (11). The miniature evaluation unit (5) could be used for housing the heating and/or cooling elements (6), the energy cell (7), a transmitter (8) and a sensor for mechanical parameters (9) and, should the occasion arise, the processing electronics.

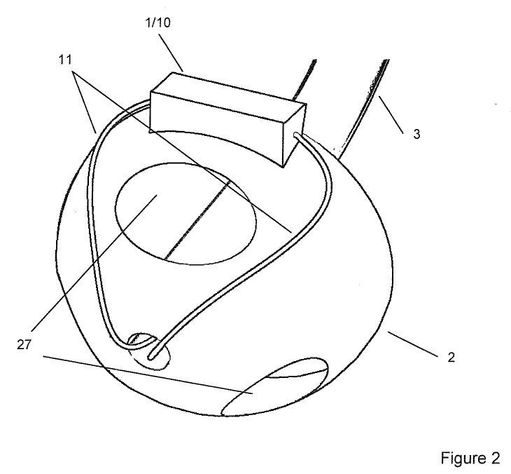

FIG. 2 shows the sensor-carrier (2) from FIG. 1 in enlarged view in its principle construction. The sensor-carrier (2) shows gaps (27) not to hinder the sound propagation. On the sensor-carrier (2) the sensor component (1) or (10) or (15) or (16) is fastened and connected with electric connection wires (11). The sensor-carrier (2) is mechanically connected with the sensor-carrier-positioning-element (3). Additionally it can be seen that the sensor-carrier positioning element (3) could be used for housing the connection wires (11).

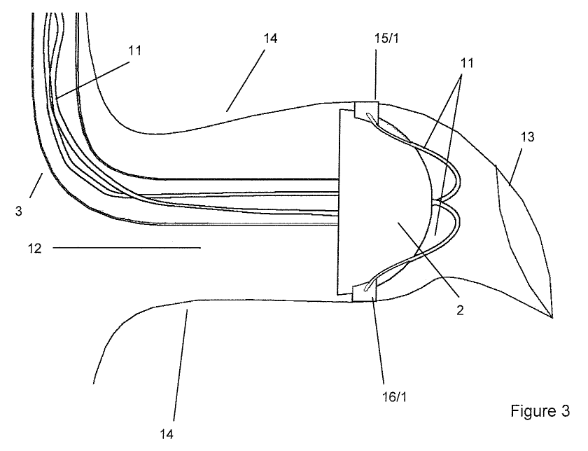

FIG. 3 shows a cross section through the external auditory canal (12) and a part of the sensor device (26) at the example of a pulse oximetry sensor. The sensor-carrier (2) is connected with the sensor-carrier positioning element (3) mechanically which carries two sensor components (1), e.g., both optical components light emitter (15) and light receiver (16). Further the electric connection wires (11) are to be seen. The sensor components (1) or (10) or (15) and (16) come to lie at the skin (14) and are in the external auditory canal (12) before the eardrum (13).

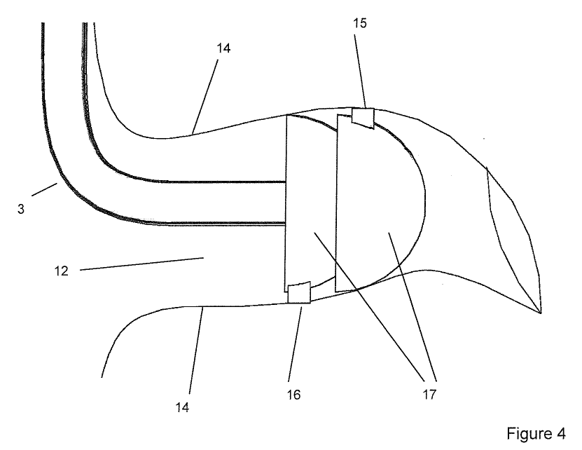

FIG. 4 shows a cross section through the external auditory canal (12) and components located in the auditory canal (12), sensor-carrier-positioning-element (3), double sensor-carriers (17), light emitters (15) and light sensors (16), specially suited for plethysmographic sensor systems, that is to say for pulse oximetry in the external auditory canal (12). One can recognize the optical separation of both optical sensor components (15) and (16) by the double sensor-carrier (17) for the blocking of shunt light (19), i.e. light from the light emitter (15) is not able to reach the light sensor (16) directly, i.e. by escaping a light path within tissue due to the optical separation by the double sensor-carrier (17). One can further recognize that the light emitter (15) and the light sensor (16) are pressed towards the skin (14) of the auditory canal (12), however shifted relative to each other in an axial direction, which results in a prolongation of the light path and the net light path (18). In addition, light emitter (15) and light sensors (16) are moved into positions radially opposing each other by about 180°.

FIG. 4 shows a cross section through the external auditory canal (12) and the components located in the auditory canal (12): sensor-carrier positioning element (3), double sensor-carriers (17), light emitters (15) and light receiver (16), in particular for plethysmographic sensor systems, i.e. pulse oximetry in the external auditory canal (12). The optical separation of both optical sensor components (15) and (16) is to be recognized by the double sensor-carrier (17) to the avoidance of shunt light (19), i.e. the light emitter (15) can reach on account of the optical separation by the double sensor-carrier (17) the light receiver (16) not directly, i.e. under avoidance of a light way in the tissue. For the increase of the light way and in particular the net light path (18), the light emitter (15) and the light receiver (16) are pressed in axial direction to the skin (14) of the auditory canal (12). Besides, the light emitter (15) and the light receiver (16) are moved around about 180° in radial direction.

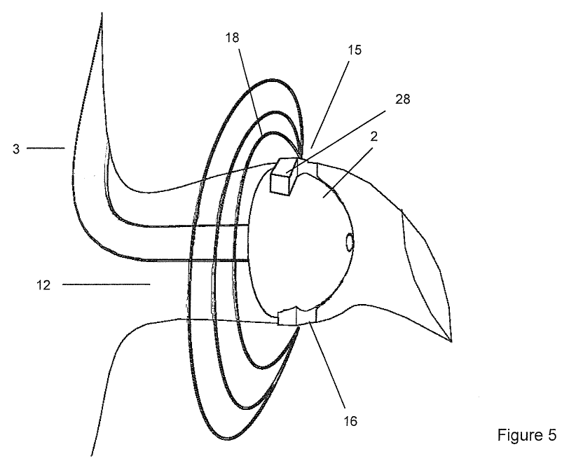

FIG. 5 shows a half-perspective cross section through the external auditory canal (12) with an optical in particular photo-metrical sensor system. Here are shown the essential light ways in the wall of the auditory canal which run in the tissue between the light emitter (15) and the light receiver (16) in a bent way due to scatter and diffraction. Out of those three exemplarily marked light ways between the light emitter (15) and the light receiver (16) the net light path (18) is special, being the most internal and shortest and most relevant one from any number of light ways traveling within the tissue. Further shown is the light receiver close to the light emitter (28) which receives a fraction of the light representative for the light emitted into the tissue (14).

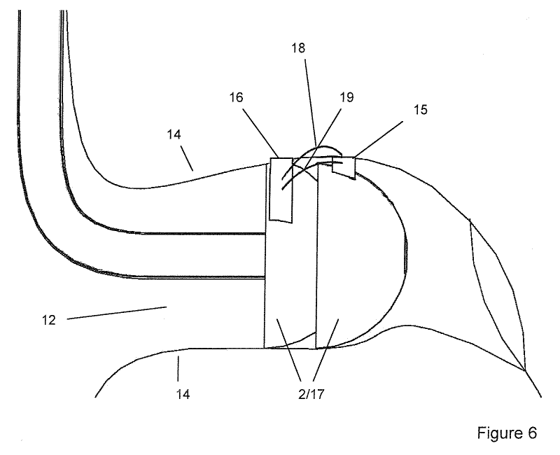

FIG. 6 shows a cross section through the external auditory canal (12) with a double sensor-carrier (17) for plethysmographic or photo-metrical sensor technology. In contrast to the FIGS. 4, 5 and 7 in this case the radial shift angle between the light emitter (15) and the light sensor (16) is clearly below 90°, e.g. 0°. As a result of this low radial shift angle between the light emitter (15) and the light sensor (16) the net light path (18) from the light emitter (15) through skin and tissue (14) of the auditory canal (12) to the light sensor (16) is relatively short, on the one hand, on the other hand, the entire optical separation between the light emitter (15) and the light sensor (16) is based on one sensor-carrier (2) only and is accordingly not quite complete, perhaps. Thus shunt light (19) arises which takes its way from the light emitter (15) directly to the light sensor (16), remaining within the auditory canal (12), that is to say without having transilluminated skin and tissue (14) of the auditory canal (12).

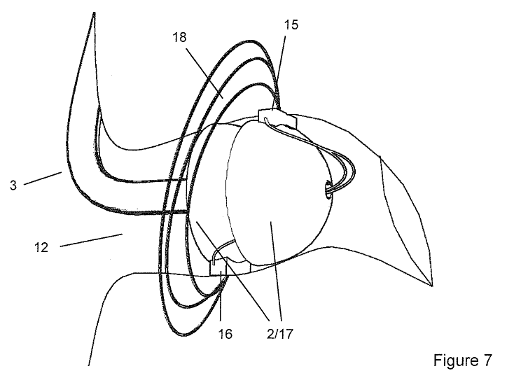

FIG. 7 shows a half-perspective cross section through the external auditory canal (12) with the plethysmographic sensor system, the sensor-carrier positioning element (3) and the double sensor-carrier (17) with the light emitter (15) and the light receiver (16). Here a double sensor-carrier (17) is used for the optical separation by light emitter (15) and light receiver (16), in addition to a nearly maximum radial blockage/separation of the light emitter (15) and the light receiver (16). Here no shunt light (19) exists and if any it is neglectably low. The marked light ways run aslant or spirally, among them the shortest and most actual one is the net light path (18). FIG. 7 shows an electric connection on the inside of a sensor-carrier (2).

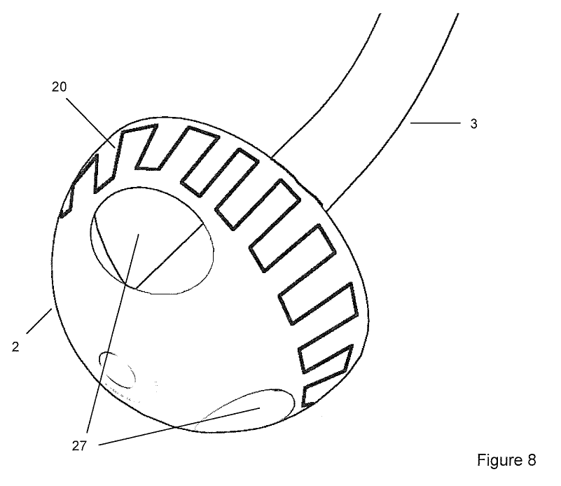

FIG. 8 shows an embodiment of a sensor-carrier (2) with an alternative method of temperature measurement. Instead of a tiny, spot-type temperature sensor, a resistor layer (20) is mounted in a meander-like fashion on the sensor-carrier (2) to measure the body(core)temperature. Thus the temperature of a broader ring-shaped area is measured within the auditory canal (12). In this way measurement errors are reduced which are due to the fact that a part of the sensor-carrier (2) does not actually touch the wall e.g. due to a crease in the sensor-carrier (2). In a ring-shaped measurement a considerable part of the temperature sensitive layer will touch the wall of the auditory canal (12); in a spot measurement just the measuring spot can not be touching. The electric connection wires (11) of the meander-like temperature sensor (20) are not drawn. The sensor-carrier (2) has holes (27) for not blocking sound propagation.

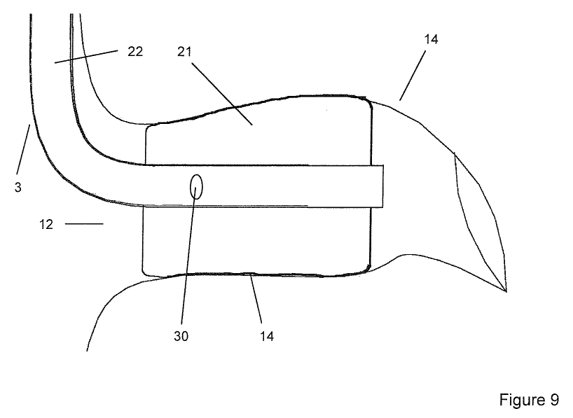

FIG. 9 shows the cross section through the external auditory canal (12) at the example of the blood pressure sensor: the expanded expansionary cuff (21), touching the skin (14) of the auditory canal (12), can be seen, here. As to this, the sensor-carrier-positioning-element (3) serves as gas- and liquid-leading connection (22). One can see a connecting orifice between the expansionary cuff (21) and the sensor-carrier-positioning-element (3).

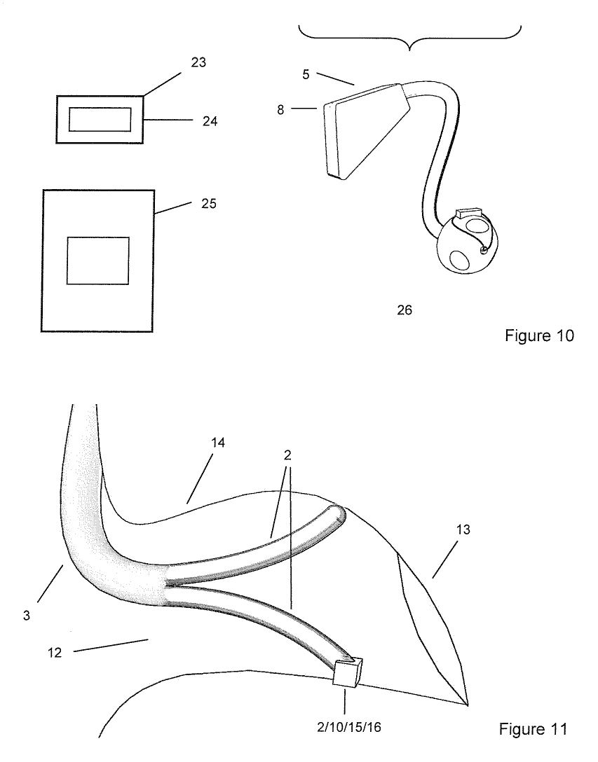

FIG. 10 shows the construction of the entire auditory canal sensor technologies comprising of sensor device (26), described in more detail in FIG. 1, and the with wires or wirelessly connected units (23), (24) and (25) in the form of modules. The modules miniature processing unit behind the auricle (5), display unit (23) or evaluation unit (24) and the mobile radio unit (25) can be, for example, spatially separate units or also be summarized at will or be completely left out.

FIG. 11 shows a 2-pole sensor-carrier (2), i.e. as an embodiment of a sensor-carrier (2) which touches the external auditory canal (12) with 2 touch points or contact surfaces. So, one can be sure, that in any case, the sensor component(s) 1 touch(es) the auditory canal. In addition, this embodiment guarantees that the sound reaches the eardrum (13) nearly unimpeded. For sensor systems which require an optical separation of the sensor components, additional measures or means may become necessary.

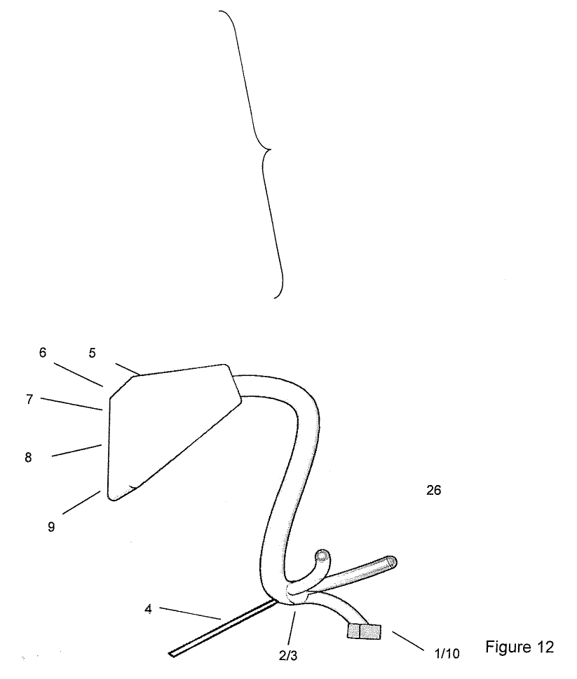

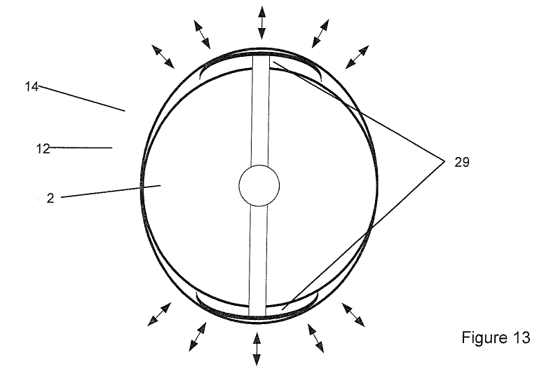

FIG. 12 shows a 3-pole sensor-carrier (2). This embodiment of a sensor-carrier 2 has a high centering effect and guarantees with high reliability that the sensor components are surely attached to the auditory canal. As in FIG. 11 the sensor-carrier (2) is permeable for sound. For sensor systems which require an optical separation of the optical sensor components (15) and (16) additional measures or means may be necessary. FIG. 12 further illustrates that there are smooth transitions between sensor-carrier (2) and sensor-carrier-positioning-element (3), i.e. there are embodiments in which would not allow a clear separation as to where the sensor-carrier (2) ends and where the sensor-carrier-positioning-element (3) begins. The terms refer to a functional concept rather than to an element of construction.

FIG. 13 shows an embodiment of a blood pressure sensor based on pressure sensor (29) instead of an expansionary cuff (21), the sensitive surface of which is kept relatively small, few square millimeters only, and bent, i.e. formed to match the internal auditory canal's wall. The pressure sensor is positioned in such a way that there is a symmetrical force distribution. Pressure sensor (29) can be also used as a pressure generator (29) in which case it applies a pressure-ramp up- and/or downward to generate the counter-pressure against the arterial pressure necessary to close the arteries and arterioles within the auditory canal, preferably according to the oscillometric blood pressure measuring principle.

### 2. Basic Components According to the Invention

The sensor component every sensor system is based on is mechanically and functionally related to the tissue in such a way that an initial signal can be derived from which the parameter can be processed as free of noise as possible. According to the invention this demand was taken care of in such a way that the sensor component as such on the one hand has a mechanical link with the tissue, as the case may be a stable positioning in form of a contact or a defined attachment pressure, on the other hand a functional link, which consists of an influence of the physiological parameter(s) to be measured or of its/their respective change on the sensor component(s) e.g. temperature or pressure, however, as well of an influence of the tissue of the auditory canal or of his anatomical or physiological components on a physical value, e.g. on the absorption of light in the auditory canal in particular on the change of the absorption by the blood vessels located in the auditory canal or by biochemical substances. Of course the sensor components are also influenced by error quantities, in mobile applications those are in particular motion artifacts, but also humidity, temperature changes etc. . . . Preferably they are removed or at least taken into consideration.

A further aspect of the invention relates in particular to the combination of the following elements:
the choice to make use of the external auditory canal as a measuring site is favorable in many respects: firstly wearing-comfort and wearing-optics are very favorable in the external auditory canal; secondly motions of the head are rather few in comparison to limbs and show a low frequency, also violent head motions are mostly felt as disagreeable and, hence, are avoided. Micromotions as they appear during talking or chewing need to be taken care of when designing the sensor while carrying the sensor and when it comes to signal analysis; thirdly the external auditory canal is well perfused which is an important condition for optic-plethysmographic measuring principles like the pulse oximetry; fourthly the external auditory canal is not included in the respiration; fifthly the external auditory canal is an area of dry skin;
the favorable positioning of the sensor component by means of the entire device comprises: sensor component, sensor-carrier, sensor-carrier-positioning-element as well as miniature processing unit behind the auricle, from which every single component contributes to the positioning as well as to the fit—firstly relative to the wall of the auditory canal (concentrically or eccentrically or wall-attached) in dependence of the parameter to be measured and secondly relative to its penetration depth;
the course of signals comprises the primary sensor system together with a low noise analog signal processing, located for example, in the miniature processing unit behind the auricle, coupled perhaps with an early analog to digital signal conversion and sending the digital information to further periphery like mobile phones, computers etc., see below.

Another common characteristic to all embodiments is that the sound reaches the tympanum mostly unimpaired by the different components of the sensor system. This is either reached, because the different components of the sensor system let the sound pass completely unimpeded or at least not significantly impeded and by the fact, that in particular the sensor-carrier is designed in such a way, that it either does not block the propagation of the sound towards the tympanum or it even actively provides such a sound propagation. To this end the sensor-carrier must not completely seal the external auditory canal or dampen it—suitable holes or gaps or channels are to be provided which can guarantee this (see e.g. FIGS. 1, 2, 8, 10). It is also possible that an active acoustic connection between the external ear and the tympanum is set up, if the measuring task makes a sound absorbing by the different components of the sensor system unavoidable. Among the rest, the active sound connection consists of a small microphone in the area of the auricle and a tiny loudspeaker in the rear auditory canal, directed towards the tympanon.

The embodiments described as follows are exemplary and differ concerning the physiological parameter to be measured and concerning the specific sensor design necessary for the respective measuring task.

The sensor-carrier is the device which positions the sensor component in the auditory canal. In many embodiments proximity and position of the sensor component to the wall of the auditory canal and the attachment force of the sensor component against the auditory canal's wall are important functional features of the sensor design for optimizing sensor performance for which the very sensor-carrier is responsible. Thus the sensor-carrier shows the property of being able to stretch or to spread respectively in order to generate the attachment force against the auditory canal's wall and to define the position of the sensor component. Hence, all embodiments have elastic or expanding properties or a shape memory, often the sensor-carrier is for example made of plastic or, eventually, of silicone and/or of a thin, springy metal. The sensor-carrier can have for example the shape of a round or an elliptical disk or a little starlet, for example, with two, three or several points of contacting the auditory canal's wall or the shape of an U- or S-shaped brace with two or more contact points or little contact surfaces but a minimum of two. Thus the sensor-carrier creates opposing attachment points, two or more, or even an attachment line but in a manner, that each attachment point has opposite counter points following a bilateral or multilateral symmetry in order to distribute the attachment forces to opposing points of the auditory canal' wall.

An especially advantageous embodiment of the sensor-carrier comprises the form of a little hood, i.e. the sensor-carrier comprising elastic or springy properties expands on the one hand perpendicularly to the auditory canal's axis until fully filling the auditory canal's circle- or ellipse-shaped cross section, on the other hand, the sensor-carrier arches over the entire circumference towards the edge in the direction of the auditory canal's axis thus generating a more or less extended tangent line, in which area sensor components can ideally be positioned: the attachment pressure is reliable and stable due to its form and is almost not depending on the spring length and is at the same time gentle and safe.

Due to the form described and due to the material properties mentioned above, the sensor-carrier centers itself in the auditory canal and thus touches the auditory canal either all around or at some points only, for example, at 2, 3 or 4 points in an attachment area each which is related with the spring tension of the sensor-carrier in such a way that the attachment pressure which the sensor-carrier applies onto the wall of the auditory canal, does not exceed a pressure of less than 40 mm of mercury (Hg), so that no circulatory disturbances appear in the tissue and in connection with it also no (hypoxia) pains when carrying the sensor for an extended period and neither any tissue damage for sure. Because the auditory canal is often rather oval than perfectly circular, non-circular sensor-carriers are also well suited. It is important that the form and the material properties of the sensor-carrier permit self-centering while inserting the sensor-carrier in the auditory canal; they even favor such a behavior.

The sensor-carrier-positioning-element is the device which defines the axial location of the sensor component in the auditory canal, i.e. its depth in the auditory canal, a value which is important for the measurement result.

The sensor-carrier-positioning-element is essential for the fit as well, i.e. it is relevant for the mechanical stability of the entire sensor device. This fit and the mechanical stability are reached by several features of the sensor-carrier-positioning-element:
by a close mostly radially effective mechanical relation with the auditory canal by means of the sensor-carrier,
by means of a narrow mechanical relation with the concave side of the auricle by means of a fixation thread and with a suitable design of the sensor-carrier-positioning-element adapting the anatomy of the concave side of the auricle
by means of a narrow mechanical relation with the miniature processing unit behind the auricle, which itself gains a grip by wedge-catch-effect against the head
by the sensor-carrier-positioning-element's material properties which cause by a form memory of the material that each partial component of the mechanical relations contributes together with the other partial components to a good over all fit of the sensor device all together and thus provides a low extent of motion artifacts.

The sensor-carrier-positioning-element is produced mainly of a tissue friendly material which possesses a form memory, e.g. one can use plastics like polypropylene or polyethylene or metals. It is typically approx. 6 to 7 cm long and has a diameter between 0.5 mm and 3 mm.

Form and length of the sensor-carrier-positioning-element and the length of the fixation thread are both preferably adapted to the individual circumstances on the one hand and the wearing side (right ear versus left ear), on the other hand, because the design of the sensor-carrier-positioning-element, as well as the insertion point of the plastic fixation thread are in mirror-image for right and left ear.

All components, including wires and fiber-optic light guides are dyed preferably skin-colored, thus a sensor built up in this way becomes completely unobtrusive.

An advantageous embodiment of the sensor-carrier-positioning-element can be structured in three essential sections based on its two typical flections within: The first bend of about 90° separates the part of the sensor-carrier-positioning-element in the auditory canal from the part which passes through the convex inner helix of the auricle upwards (cranially). The second bend of more than 90° runs around the upper turn over line of the auricle towards the miniature processing unit behind the auricle. This miniature processing unit is preferably adapted in its form to this space in a way that it provides sufficient space for power supply and electronics, but also fits well there and finds a good grip between auricle and scalp and gets firmly caught there.

The fixation thread leaves the sensor-carrier-positioning-element more or less right-angled and is flexible to fit into the rear curvature of the convex helix of the auricle. Thus additional fit is provided for the sensor-carrier-positioning-element and along with it for the entire sensor device. The fixation thread consists of a round, flexible, material; a solid material plastic thread can be used or a plastic tubing with a diameter of 1 to 2.5 mm for example.

As to this end the sensor-carrier-positioning-element is equipped to engage with the sensor-carrier into a form-locking, separable connection, e.g., a spring type connection or snap-in connection i.e. a small thickened section on the sensor-carrier-positioning-element and a small reinforced (center-) opening in the sensor-carrier are adapted to each other that they can “engage” into each other. This optimum auditory canal's depth depends on the anatomical circumstances, so that for this length of the sensor-carrier-positioning-element some typical lengths must be kept at hand. The sensor-carrier with the sensor(s) is then form-fittingly connected with the best-fitting sensor-carrier-positioning-element.

The sensor-carrier-positioning-element is mostly shaped like a little tube or pipe, i.e. hollow inside, and thus sets up a mechanical and/or optical and/or electric connection and/or pressure-conducting connection (e.g. air/water) between the sensor component on the one hand and the miniature processing unit behind the auricle, on the other hand, and, for example, the fiber-optic light guides or the electric wires come to lie inside of the sensor-carrier-positioning-element and thus transfer signals and potentials.

However, the sensor-carrier-positioning-element can also serve as a guiding structure, for example, for the connecting wires of the sensor component or the light- gas- or liquid-guides respectively. For example, the connecting wires can be extruded within the plastic or together with it or be connected in another way with the sensor-carrier-positioning-element. If wires are guided through the sensor-carrier-positioning-element, they can enter e.g., in the auditory canal, near the little hood into the sensor-carrier-positioning-element and then leave the sensor-carrier-positioning-element inside the miniature processing unit behind the auricle.

In addition there can be transitions, blending into each other between sensor-carrier and sensor-carrier-positioning-element, i.e. there are embodiments in with no clear demarcation between these both components is to be recognized. In this case that component, which extends into the auditory canal, should be considered as the sensor-carrier-positioning-element, that which carries the sensor component as the sensor-carrier, no matter whether an external demarcation is possible. Then it is a functional differentiation, which counts.

The miniature processing unit behind the auricle at the end of the sensor-carrier-positioning-element looking away from the sensor component is an element of mechanical fixation of the entire sensor device due to its good fit as a result of its “wedge-catch-effect”. In a preferred embodiment it contains the energy cells as well as if necessary a part of the electronic components for signal processing. It also contains if necessary electronics for the signal forwarding, e.g., via infrared or by radio, e.g., via Bluetooth or Zigbee or another realization of a wireless, digital or analogous transfer function. As a result of the place restriction the miniature processing unit behind the auricle can be just a telemetry unit as well, i.e. signals sent from it are forwarded to the true evaluation unit and/or memory unit and processed further before they can be used by the user. The miniature processing unit behind the auricle must not imperatively contain the described components, it can be totally empty as well and serve exclusively as a functional component for the fit of the sensor device. For the functionality according to the invention it is insignificant whether the respective modules such as the miniature processing unit behind the auricle as well as the display unit or the evaluation unit and the mobile radio unit really are spatially separate units. According to desired functionality and according to achievable miniaturization these modules can be summarized or be also completely left out at will.

In the following embodiments are exemplarily disclosed under functional points of view. The embodiments are designed for certain measuring tasks or certain bio-parameters especially.

### Parameter Body(Core)Temperature:

Platinum resistor probes, e.g., Pt100, PT500 or Pt1000 resistor temperature sensors are especially well suited because of their widely linear and relatively steep characteristic, but NTCs are also suited. In order to measure the body(core)-temperature the following features of the embodiment(s) should be considered: one or several tiny temperature sensors based on convection i.e. contact-sensors are positioned on the sensor-carrier in a way, that they preferably touch the middle or rear auditory canal in a thermally conducting way. Platinum resistor sensors, e.g., Pt100, PT500 or Pt1000 resistor temperature sensors are well suited because of their widely linear and relatively steep characteristic curve, on the other hand NTCs are also suited.

An advantageous embodiment shows a design with several temperature sensors positioned on the sensor-carrier in order to receive several temperature data. Because temperature always is a distributed quantity temporally and spatially, a suitable temperature needs to be selected. This could be, for example, the maximum temperature, because it nicely correlates with the body(core)temperature. However, one can conclude the quality of the measuring situation, among other things the quality of the thermal contact from the temperature distribution of the single sensors.

In principle one can also use a radiation sensor which is positioned on the sensor-carrier and whose angle of incidence is typically aimed at the internal part of the auditory canal, i.e. the tympanum and the surrounding/inner auditory canal.

A further embodiment consists in using a thin platinum layer as a temperature sensor which is positioned, for example, in a meander-like fashion onto the edge of the little hood. This meander-like resistor layer could fully extend around the circular touch strip, or just around some part of it.

If a convection sensor is selected as a measuring principle, the temperature sensor in any form, as a compact, miniaturized sensor of any technology, as well as a layer sensor needs to be pressed against the tissue with an attachment pressure so low that no irritations in the tissue are caused, but large enough to be able to produce a reliable thermal contact with the tissue.

For the measuring kinetics it is favorable if the temperature sensor per se has a small thermal mass and if this thermal mass does not considerably grow as a result of adjoining materials, e.g., by the sensor-carrier. This can be reached either by thermally isolating the temperature sensor from the material of the sensor-carrier, or by taking care, that the sensor-carrier itself has a low heat capacity and/or heat conduction. In addition a good thermal contact with the wall of the auditory canal is important. By the measures described above, it can be achieved that the body has a great deal of influence; the influence of the environment however remains small.

A frequent source of error is the exchange of air in the auditory canal by ambient air. According to the invention this source of error can be counteracted in many ways: Firstly this problem becomes the lower and the measured temperature resembles corresponds the more exactly to the body (core) temperature, the further inside the auditory canal the sensor is positioned, the less the temperature is contaminated by air from the outside. Secondly the air exchange with ambient air can be reduced e.g. by sealing the external auditory canal a little, for example, by means of a porous stopper, or by covering the auricle more or less loosely, for example, with an ear protection, headscarf, headband or a cap. Thirdly one can determine the error quantity: for this purpose one measures the temperature gradient to the environment by use of several sensor components and takes this information for the correction of the error quantity. Fourthly one can reduce the error quantity: for this purpose a device is attached in the external area of the auditory canal which reduces the gradient between the body(core)temperature and the temperature of the outside world e.g. a kind of a heating or a cooling, according to the direction of the gradient. The measurement error drops with the reduction of the gradient.

The measurement the body(core)temperature in the ear turns out to be the more exact, the deeper the sensor component is positioned in the external auditory canal. As to this, however, limits are involved, because any touch becomes the more disagreeable in the external auditory canal, the more the tympanum is approached.

For the use of the external auditory canal for the measurement of the body(core)temperature, the attachment pressure of the contact sensor against the auditory canal is a prerequisite. Should it be missing, e.g., due to an incorrect insertion of the sensor, this can be recognized and taken into consideration according to the invention preferably by means of the heat capacity and/or heat conduction of the matter surrounding the sensor component: air has a low thermal conductivity or heat capacity, tissue has a high thermal conductivity or heat capacity. For the differentiation of the surrounding matter the sensor component is brought to an excess temperature, i.e. a heating phase is inserted into the measurement of the body temperature during which the surrounding matter is brought to an excess temperature. Immediately after the heating phase (measuring phase) the temperature or the decrease in temperature is measured. From the kinetics of the heat loss one can conclude on the surrounding matter, because air cools down much faster than tissue. The use of just one sensor resistor for all heating and measuring tasks is neat.

### Parameter Blood Pressure:

According to the invention the external auditory canal is used as a measuring site for measuring the blood pressure. According to the invention either mechanical or optical methods are taken into consideration for this. As to this the sensor design is the essential inventive feature, whereas the method for measuring the blood pressure can be in accordance with the state-of-the-art. Since due to the low tissue layer thickness of the auditory canal's wall only low plethysmographic volume shifts occur i.e. small pulsating blood volumes ΔV, effects referring to volume shifts are very small. Hence, pressure-based measurements are to be preferred, because principally the same blood pressure conditions apply in the auditory canal as well.

In an important embodiment the tissue of the external auditory canal is compressed centrifugally. In contrast to blood pressure measurement cuffs on limbs which contract towards the central axis, the compression takes place here off the central axis towards the outside, by means of an expanding cuff, i.e. a roller-shaped balloon which is expanded against the external auditory canal by filling it with a gas (mixture), more advantageously with a liquid. The blood pressure measurement cuff in the external auditory canal works expansively. The volume of the expanding cuff should be small, so that its compression remains measurable. Secondly incompressible media are preferred for the forwarding of the intra-arterial pressure changes. Thirdly as far as possible all parts of the expanding cuff which are not in touch with pulsating parts of the body should be rigid, so that the whole plethysmographically shifted blood volume ΔVₚₗₑₜₕ is transferred to the measurement cuff and is converted into a measurement volume ΔVₘₑₛₛ which then generates the measuring effect as a pressure change or as a volume change or as an other measured variable and so that no losses occur. Losses occur, for example, if in the side areas of the measurement cuff a fraction of the measurement volume ΔVₘₑₛₛ is used up or lost due to the fact that cuff-walls or flexible hose walls are deformed or gases are needlessly compressed. Thus in a preferred embodiment of such an expanding cuff this is malleable only in the area in which it touches the auditory canal. For the measurement of the pressure in the expanding cuff either a pressure measuring device is already positioned within the expanding cuff, or the pressure is passed on to a pressure measuring device, for example, through the sensor-carrier-positioning-element which should be relatively pressure-stable for this purpose of course. The sensor-carrier-positioning-element serves as a device for the correct positioning of the sensor component, for example, of the expanding cuff and if necessary of a pressure measuring device in its core or immediately next to the cuff and/or serves the purpose of further propagating the signals, e.g. the electric signals of a pressure measuring device, or if necessary of creating a connection for gas- or liquids with the pump and/or with the pressure measuring device which are positioned, for example, in the miniature processing unit behind the auricle.

A further preferable embodiment of such a blood pressure measuring sensors consists of the use of small detector surfaces, that is to say, small pressure measuring means which touch the auditory canal in tiny flat areas which subtend each other or are positioned in 120° angles in such a way that the sensors center themselves, or that each pressure sensor relates to an opposing point/to opposing points i.e. has its foot on (the) opposite wall(s). It is advantageous, if the elements which can measure the pressure and the elements which exercise the (occluding) pressure upon the wall of the auditory canal, in order to compress the arterial vessels up to a closing pressure and beyond, are positioned close to each other or are identical.

In a further embodiment according to the invention the excitation or the pressure changes as a function of the pressure upon the auditory canal's wall that is to say the oscillometric information is/are derived optically.

The monitoring of the blood pressure is preferably derived from both of the information pieces, pressure upon the auditory canal's tissue, on the one hand, and the relative pressure amplitude which is transferred from the tissue's arteries and arterioles, oscillating synchronously with the heart, to the pressure measuring system, on the other hand, i.e. it is based, for example, on the oscillometric measurement technology. The systolic pressure can also be derived, for example, by optical means for example by means of suppression of optic-plethysmographic phenomena, the systolic pressure can be identified as the pressure at which arteria are blocked. In addition the necessary optical components can be integrated into the expanding cuff or be placed in its immediate surrounding.

A special embodiment plans to use the expanding cuff also for a temporary protection of the ear against excessive noise, simply by blocking the auditory canal by means of expansion.

When calculating the blood pressure detected in the auditory canal the (negative) hydrostatic pressure which results from the distance of the external auditory canal relative to the heart is to be taken into consideration. As to this it is useful to provide a button or a switch with which the user can transfer to the blood pressure measuring instrument the position: either vertically (=standing, being seated) or horizontally (=lying).

A favorable embodiment intends that this information is detected by the miniature processing unit which contains for this purpose at least one, preferably several inclination sensors.

An advantage of this measurement technology is that the user can not receive erroneous measurements on account of a variable hydrostatic pressure as it is the case, otherwise, with varying positions of the measuring arm relative to the heart. This requires, however, that a single calibration had been performed, which takes the distance of the external auditory canal to the heart into consideration.

The measurement of the blood pressure can be performed making use of the device or of the method according to the invention in peace and quiet as well as under mobile conditions. The measurement in peace and quiet allows in particular a screening concerning the diagnosis of (essential) hypertension and the assessment of antihypertensive therapeutics as well. The diagnostic importance of the parameter blood pressure under mobile conditions, in particular under stress can hardly be estimated, even today. Thus the measurement of the blood pressure, e.g., doing sport could allow an assessment of the training success and of certain strain or exhaustion conditions. The water balance is also reflected in the blood pressure profile. In the end, the blood pressure and the heart rate, perhaps, also in combination with the body(core)temperature are also components of an assessment of the vigilance what is of considerable importance especially for drivers of motor vehicles.

### Parameter ECG:

The monitoring of electric potential differences, e.g., ECG, can be performed according to the invention at different places in or at the ear. At least one electrode is positioned in the auditory canal on the sensor-carrier in such a way, that a reliable electric contact with the wall of the auditory canal is established. A further electrode is placed, e.g., behind the auricle and is, for example, attached to the miniature processing unit behind the auricle. The greater the spacing of the first electrode from the second one, the greater is the potential difference and the better the S/N. p. ratio. Accordingly the second electrode could be also positioned contralaterally i.e. at the other ear, or better at an arm, for example designed like a wristwatch or even better in (electric) proximity to the thorax.

Electric potential differences of other origin could be monitored in the same manner.

A favorable embodiment of an auditory canals electrode consists of a circular conductive layer on a round or hood shaped sensor-carrier which touches the auditory canal at as many spots possible, similar to the meander-like temperature sensor layer of the sensor component for measuring the body temperature, however, without the high temperature coefficient.

### Measurement of Photo-Metrical and Optic-Plethysmographic Values:

The advantages of using the external auditory canal as the site of measurement, e.g., for pulse oximetry instead of using a finger's distal phalanx are multiple ones: the optical sensors are comparatively well protected inside the auditory canal form ambient light, i.e. in comparison to a finger sensor it is to be reckoned on less foreign light. In addition lower accelerations appear at the head than at a limb, and even much less than at a finger which has an exceptionally good mobility. However, another decisive advantage of the measuring site “auditory canal” is that an auditory canal's sensor clearly means less irritation or impediment than a finger sensor. It is of importance, too, that a well designed and extremely miniaturized auditory canal's sensor hardly attracts attention, it is hardly noticed. It is even possible, that a trend can be established, i.e. an auditory canal's sensor will be equipped with suitable stylish decorative features, so that it is considered to be “trendy” and is thus even worn with pleasure.

Miscellaneous values or parameters to be measured need different sensor components. Within the scope of the invention not only embodiments are disclosed which have the sensor component positioned in the auditory canal, but also embodiments which have the sensor component positioned outside the auditory canal, however, the physical measured variable derived from within the auditory canal is associated with the sensor component outside the auditory canal. Accordingly light can be generated, for example, by LEDs outside the auditory canal, for example, in the miniature processing unit behind the auricle and be conducted onwards into the auditory canal by means of fiber-optic light guides or vice versa light from the auditory canal is conducted to a light sensor outside the auditory canal via fiber-optic light guides. The principle of the invention remains intact from questions like as to where the optical components are positioned—the place at which the tissue optical measurement layer is located remains the auditory canal.

Which sensor components, are operated in detail for which measurement variable ever, attention needs to be paid that the thermal power loss in the auditory canal is not exceeding temperatures of 42° C. at any place to avoid tissue damages following fairly long term exposure to the warming. As a recommended value which should not be exceeded, a thermal power loss of about 30 mW continuous small environment tissue exposure could be found in own measurements as it is the case e.g. when using LEDs within or attached to tissue for pulse oximetry.

According to the invention optically accessible parameters can be monitored in the external auditory canal as well. For the measurement of photometrical and optic-plethysmographic values e.g., pulse oximetry according to the invention, the subcutane tissue of the external auditory canal is transilluminated. In this respect it is advantageous that the tissue of the external auditory canal shows sufficiently high perfusion so that high modulation depths originate.

The external auditory canal is virtually looked at as a cuvette with a defined layer depth whereas the layer depth corresponds to the net light path. The external auditory canal is further looked at as a cuvette for substances which are dissolved in the blood of the surrounding tissue.

It is known, that in tissue photometry/pulse oximetry the absorption of light mainly the variable absorption of light in time that is to say the modulation is determined in particular in a certain frequency range, for example, from 0 to 0.5 hertz as well as 0.5 to 10 hertz which is related to the smallest or biggest observable pulse rate or respiration rate, and the blood pressure and other quantities of the organism to be examined.

The light flow in the tissue is caused in particular by different optical effects like scatter, absorption, diffraction among other things at bordering layers between different components of the living tissue, whereas “transmission” and “reflection” are simplifying terms which rather refer to a microscopic-geometrical behavior of the light relative to transmitter and receiver.

Thus dealing with the optical plethysmography the absorption of light, in particular the modulation of light is observed in two different frequency spectra:
in the so-called pulsatile spectrum, the alternating light spectrum, also referred to as AC, in the frequency range of the heart rate, the arterial pulse, i.e. from approx. 30 to 240 bpm, corresponding 0.5 to 4 hertz (maximum range) or from approx. 40 to 150 bpm, corresponding from 0.67 to 2.5 hertz (normal range) or also in the frequency range of the respiration rate from 5-20 per minute and
in the so-called steady, non-pulsatile spectrum, the constant light spectrum, also referred to as DC, in the frequency range of less than 0.5 hertz wherein “constantly” and “non-pulsatile” is meant in relation to the heart rate or even in relation to the respiration rate.

The division of AC by DC reveals the modulation depth MD for the mono-chromatic light or for the light spectrum, respectively:

$${MD}_{{λ}_{x}} = \frac{{AC}_{{λ}_{x}}}{{DC}_{{λ}_{x}}} ,$$

Thus, it is possible to monitor among others, the pulse rate and the respiration rate, eventually also the blood pressure and other bio-parameters.

If an optical plethysmography is carried out in two or more spectral ranges (λ1, λ2 . . . λn) and the respective modulation depths (pulsatile part, AC, relative to the non-pulsatile part, DC) are related, e.g., with 730 nm and 880 nm to each other, a variable omega is derived from the respective modulation depths, (in English: ratio)

$$Ω = \frac{{MD}_{{λ}_{1}}}{{MD}_{{λ}_{2}}} , Ω = \frac{{( \frac{AC}{DC} )}_{{λ}_{1}}}{{( \frac{AC}{DC} )}_{{λ}_{2}}}$$

which is widely independent from factors of influence like the layer thickness of the transilluminated tissue or the strength of the light emitters etc. and with the help of which the arterial oxygen saturation can be investigated, eventually the venous oxygen saturation as well, perhaps, also the arterio-venous saturation difference.

The spectroscopic background is that human hemoglobin exists in the essentials in two conditions, namely as oxygenated hemoglobin, HBₒₓ, and as desoxygenated one, HB_{red}, if one refrains from toxically changed or genetically deviating hemoglobin fractions.

HBₒₓ and HB_{red} show different specific spectral absorption curves of which in particular the area between 600 nm and 1000 nm can be used for the purposes of the optical plethysmography. In addition one measures the absorption of the light by both hemoglobin factions (oxygenated and deoxigenated) in two different spectral ranges, more exactly one measures the light intensity after the passage through tissue in which arterioles pulsate in which blood with both hemoglobin fractions flow.

For the invention related measurement of photo-metrical and optic-plethysmographic quantities again components according to the invention like sensor-carrier, sensor-carrier-positioning-element as well as the miniature processing unit behind the auricle are provided as basic components of the sensor design for the application i.e. for positioning, placing, arranging of light emitters and light sensors in particular for the production of suitable light paths. Embodiments of photo-metrical or optic-plethysmographic auditory canal sensors, that is to say, of sensors for pulse oximetry, for plethysmography, or for the monitoring of substance concentrations show the following essential, advantageous characteristics concerning the sensor design:
A light path through the wall of the external auditory canal, i.e. through the tissue of the auditory canal's wall is generated. To this end the light which is generated by at least one, mostly by several light emitters is radiated into the tissue of the auditory canal and is then emitted at another place and detected by a light sensor. In this fashion absorption of light in the tissue is guaranteed, only in this fashion the optical properties of the tissue or of the blood located in the tissue or of substances located in the blood can be monitored.
The avoidance of shunt light is essential, because this leads to measurement errors. According to the invention particularly suited to blocking shunt light are light-impervious disks or little hoods which expand in the auditory canal and thus divide light emitter and light sensor into two optical half spaces separate from each other. Accordingly light can reach the light sensor only through the tissue. It is especially advantageous if the sensor-carrier helps towards the suppression of shunt light in the wider sense, i.e. one or several light-impervious sensor-carriers block the direct light path. The positioning of the light emitter on one side and the positioning of the light sensor on the other side of an optically impervious little hood-shaped or disks-shaped sensor-carrier already prevents the direct light path. If several sensor-carriers are staggered the safety of shunt light suppression can still be improved.
To receive a good photo-metrical or optic-plethysmographic signal respectively, in particular to receive a high modulation depth, it is important that the light travels a very long way within the tissue on its way from the light emitters to the light sensor, that the net light path is very long, ideally even longer than the longest geometrical light path.
Light emitters and light sensors are preferably positioned opposite to each other, facing outwardly, i.e. 180 relative to a circular sensor-carrier, shaped for example like a disk or a little hood. Thus, both optical components are positioned maximally separated. Accordingly the light is radiated into the skin of the auditory canal and is emitted again on the opposite side, after having traveled a net light path of a semicircle shape through the tissue of the auditory canal, more exactly: two semicircles having axial symmetry where “axis” means the connection between light emitter and light sensor. In particular due to the symmetry the maximum distance arises in 180° constellation, i.e. the longest net light path and with it also the best optic-plethysmographic signal and, above all, the highest modulation depth—real transmission pulse oximetry in the narrowest sense.
Also an embodiment is successful, in which light emitters and light sensors do not precisely face each other (rotation in the sensor-carrier's plain: 90° to 180°). Here the light intensity in the shorter light path predominates, i.e. the shorter arc and therefore the partial component with the higher light intensity dominates the plethysmographic signal. Such a non-maximum light path seems a logical choice if the semicircle light path was too long to be transilluminated with tenable effort. On the other hand a shortening of the light path includes the danger to have shunt light involved.
Possibly one sensor-carrier is not sufficient to completely block the shunt light. In a further embodiment according to the invention two or more sensor-carriers are positioned one after the other on the sensor-carrier-positioning-element. Light emitters and light sensors can be differently arranged relative to each other, as described for one sensor-carrier, it's best to have them maximally separated, i.e. positioned 180° relative to each other. As a net light path the result is again a semicircle, however, slanting through tissue, from one sensor-carrier to the opposite position on another one. This net light has the maximal length, maximizes the modulation depth accordingly and minimizes at the same time the shunt light influence—a real transmission pulse oximetry in the external auditory canal is reached, in the end.
Especially favorable is an embodiment with maximally separated optical components on two or more sensor-carriers, in the end, because several light-impervious sensor-carriers block the shunt light particularly reliably.
In a further embodiment the light emitter and light sensor lie more or less side by side (rotation in the sensor-carrier's level: 0° bis 90°)—in the end, a reflex pulse oximetry in the external auditory canal. More than one sensor-carrier is necessary for this embodiment: light is then transmitted from the location on one sensor-carrier to the adjacent location on the neighboring sensor-carrier through the skin of the external auditory canal. By use of this embodiment the light intensity rises, on the other hand shunt light increases considerably, whereas the modulation depth drops. Such a light path seems appropriate if spectral ranges are used in which the specific spectral absorption is so high that only very short light paths can be realized with tenable effort.
For the monitoring of photo-metrical values at least one further light sensor can be positioned optically immediately adjacent to the light emitter, so that additional information about the light intensity entering into the tissue is available. This information is especially important for the monitoring of absolute concentrations of substances in the tissue or of substances in the blood which is passing the tissue. According to the principles of the photometry not only the light intensity I must be known after the passage through the tissue, but also the input light intensity Iₒ i.e. the light intensity radiated into the tissue.
It is advantageous if light emitters and light sensors are positioned in any case in the periphery of one or more sensor-carrier(s) radially turned outwardly, the respective directions of the optical sensor components being allowed to be different of course.
An alternative light path's guide consists in simply emitting the light into the space of the auditory canal, accordingly only the light sensor is turned towards the tissue or is actually attached to it. Vice versa the receiver can receive light from the space and the light emitter is attached to the tissue. A certain disadvantage of this embodiment is that thus the net light paths are not clearly defined.

A certain attachment pressure of the optical sensor components against the tissue is favorable in general. As to this, it has to be considered, however, that the attachment pressure has an optimum, i.e. not enough or too much attachment pressure is equally unfavorable. Varying attachment pressure conditions are especially unfavorable, such as motions at the sensor generate them or motions of the sensor relative to the tissue—they generate motion artifacts. In order to suppress such motion artifacts in the best way possible or better still to avoid them completely, the sensor-carrier-positioning-element should transfer as few motions as possible, in particular no motions in an axial direction. In respect to the design, motions can be reduced by integrating a device into the sensor-carrier-positioning-element which absorbs motions or allows their compensation. For the reduction of axial motion artifacts a device would be suitable which absorbs push and pull motions similar to a telescope element. Also tiny motion detectors could sense motions and thus enable that they are recognized and suppressed by means of signal-processing.

The demands on the sensor-carrier, however, to be optically impervious on the one hand, but acoustically permeable on the other hand, are not trivially compatible with each other. As solutions according to the invention the following basic concepts are suggested: The first embodiment comprises a sensor-carrier made of light-impervious material, but permits the propagation of sound in the auditory canal, either by its high ability to oscillate based on the material or by sound conducting but light absorbing structures such as small bent tubes or a few, propitiously staggered gaps. A further embodiment comprises a sensor-carrier made of light-impervious material without any holes; the propagation of sound occurs actively, i.e. by means of a tiny microphone in the distal area of the external auditory canal or in the auricle and a tiny loudspeaker near the drum respectively.

### Measurement of Mechanical Values Like Position, Acceleration and Location:

According to the invention mechanical sensors are positioned either in the external auditory canal, or in the miniature evaluation unit behind the auricle, on the one hand per se or, on the other hand, next to sensors for other parameters. Sensors for the measurement of mechanical parameters are, for example, inclination sensors, acceleration sensors for linear motions or rotation, sensors for the position of the head relatively to other body parts, for example, body and limbs, or location sensors, such as for example GPS or the like. Thus states like physical activity, positions like standing position, sitting, lying and their change(s), e.g., falling, danger of falling, accident, sit down, lay down, stand up can be detected, as well as physical efforts like running, stamina, training degree, can be estimated and indicators for sleep, sleep phases and sleep quality and many others more can be obtained. This information is important as such. It becomes more significant in connection with other parameters such as heart rate, respiration rate or oxygen saturation, the interpretation of which they considerably improve and extend: Blood pressure during physical exercise, sports, respiration during the sleep, body(core)temperature during sports, training and rehabilitation etc. According to the invention information about position and motions is important for the interpretation of sensor data in a medical overall context. Moreover, data about position and motions allow the appraisal of disturbance variables. Thus in advantageous embodiments disturbance variables are measured and are used to correct the usable information or to make use of the usable information only if the measurement is performed under low noise conditions.

Disturbance variables can be mechanical and thermal influences, and can also derive from the immediate anatomical environment: chewing, yawning, coughing, as well as consuming warm and cold dishes and drinks. However, these disturbance variables are of a passing nature and can be kept out of a continuous measurement as “high-frequency signals” by use of signal processing means. On the other hand, it can be in the interest of certain users to study exactly these influences.

### REFERENCE NUMBER LIST

1 sensor component
2 sensor-carrier (e.g., singles, double or multiple little hood)
3 sensor-carrier positioning element
4 fixation thread (for the insertion into the convex helix of the auricle)
5 miniature evaluation unit behind the auricle
6 heating and/or cooling element (in miniature evaluation unit behind the auricle)
7 energy cell (in miniature evaluation unit behind the auricle)
8 transmitter (in miniature evaluation unit behind the auricle)
9 sensor for mechanical parameters (e.g. in miniature evaluation unit behind the auricle)
10 temperature sensor (sensor component)
11 electric connection wires
12 (external) auditory canal
13 eardrum
14 skin and tissue
15 light emitter (sensor component)
16 light receiver (sensor component)
17 double sensor-carrier (double hood)
18 net light path
19 shunt light
20 temperature sensor with metal layer meander
21 expansionary cuff
22 connection for the transport of gas or fluids
23 display unit
24 processing unit (e.g., in concealed, sewed-on jacket)
25 mobile radio unit
26 sensor/sensor device
27 gap in the sensor-carrier for the sound propagation
28 light receiver near the light emitter (sensor component)
29 pressure sensor/pressure generator
30 pressure equilibrium hole

Now that the invention has been described, I claim:

1. A device for the measurement of at least one bio-parameter selected from physiological and/or biochemical and/or bioelectric parameter, comprising:
a sensor-carrier (2) adapted to being placed in the auditory canal,
at least one sensor component (1) for measuring at least one parameter when placed in the auditory canal, said sensor positioned on and/or connected with which sensor carrier,
a sensor-carrier-positioning-element (3) connected with or merging into the sensor-carrier (2), and
electronics integrated in the device for the measurement of at least one parameter.

2. The device according to claim 1, wherein the sensor-carrier-positioning-element (3) determines the penetration depth of the sensor-carrier (2) in the auditory canal and is held in the auditory canal by the sensor-carrier (2).

3. The device according to claim 1, wherein the sensor-carrier-positioning-element (3) is designed to determinate and optimize the penetration depth of the sensor-carrier (2) in the auditory canal, and the sensor-carrier (2) is designed to determinate and optimize the attachment force to the auditory canal and/or the net light path of the sensor component (1), and the sensor-carrier-positioning-element (3) comprises means for mechanically stabilizing the entire sensor device (26) thereby avoiding or minimizing motion artifacts.

4. The device according to claim 1, wherein
the sensor-carrier-positioning-element (3) determines and optimizes the penetration depth of the sensor-carrier (2) in the auditory canal (12),
the sensor-carrier (2) determines and optimizes the attachment force and/or the net light path of the sensor component (1), and
the sensor-carrier-positioning-element (3) comprises means for mechanically stabilizing the entire sensor device (26) thus avoiding or minimizing motion artifacts.

5. The device according to claim 4, wherein the means for mechanically stabilizing the sensor device (26) for avoiding or minimizing motion artifacts comprise:
means of anatomical adaptation of the sensor-carrier-positioning-element to an auricle, such as the fixation thread (4) and/or bends in the sensor-carrier-positioning-element (3)
the miniature evaluation unit's stabilization properties by wedge-catch-effect between the auricle and the skull, and
the sensor-carrier's fitting means providing a stabilizing mechanical relationship to the (external) auditory canal (12).

6. The device according to claim 3, wherein the means for mechanically stabilizing the sensor device (26) for avoiding or minimizing motion artifacts comprise at least one of:
means of anatomical adaptation of the sensor-carrier-positioning-element to an auricle, such as a fixation thread (4) and/or bends in the sensor-carrier-positioning-element (3),
provision of a miniature evaluation unit with stabilization properties such as wedge-catch-effect between the auricle and the skull, and
the sensor-carrier's fitting means providing a stabilizing mechanical relationship to the (external) auditory canal.

7. The device according to claim 1, wherein the parameter is selected from the group comprising the body temperature, the oxygen saturation of the blood especially the arterial blood, the heart rate, electric parameters of the heart (ECG), the respiration rate, the blood pressure, the concentration of substances dissolved in the blood, the concentration of substances present in the tissue, the physical activity, mechanical parameters of the body and parameters in relation to sleep and vigilance.

8. A device for monitoring tissue optical quantities, mainly for monitoring the oxygen saturation in the arterial blood, in particular for the realization of pulse oximetry based on a sensor in the auditory canal, comprising at least one sensor-carrier (2) with a sensor component (1) in form of at least one light emitter (15) and of at least one light receiver (16),
wherein the device includes means for preventing that light travels directly from the light emitter (15) to the light receiver (16) without transilluminating the tissue (i.e. means for avoiding shunt light).

9. A device according to claim 1 for monitoring tissue optical quantities, mainly for monitoring the oxygen saturation in the arterial blood, in particular for the realization of pulse oximetry based on a sensor in the auditory canal, designed such that the light path through the tissue of the auditory canal follows the principles of Circummission Pulse Oximetry.

10. A device according to claim 8, wherein the means for preventing that light travels directly from the light emitter (15) to the light receiver (16) without transilluminating the tissue is a clamp shaped, disc-shaped or umbrella-shaped element in which the light emitter (15) is positioned on one side and the light sensor (16) on the other side, or an umbrella-shaped double sensor-carrier (17) as to which the light emitter (15) is positioned on one umbrella and the light sensor (16) in the other umbrella.

11. A device for the monitoring of tissue optical quantities, mainly of the oxygen saturation in the blood, in particular for the realization of pulse oximetry in the auditory canal, comprising at least one sensor-carrier (2) with a sensor component (1) in form of at least one light emitter (15) and of at least one light receiver (16),
wherein for the purpose of the maximization of the light path length or to the suppression of shortened light paths when said device is placed in the auditory canal, means is applied of heavily light-diminishing mainly light-blocking cover of more than half of the auditory canal's half-circumference in the area of the net light path (18) to both sides of the light sensor (16), mainly in form of an arched, umbrella-shaped sensor-carrier (2) or of several such sensor-carriers (17).

12. A method for the monitoring of tissue optical values, mainly of the arterial oxygen saturation in the blood, in particular for the realization of pulse oximetry in the auditory canal, comprising:
(a) introducing into an auditory canal a device comprising a sensor-carrier (2) adapted to being placed in the auditory canal, at least one sensor component (1) for measuring at least one parameter when placed in the auditory canal, said sensor positioned on and/or connected with which sensor carrier, a sensor-carrier-positioning-element (3) connected with or merging into the sensor-carrier (2), and electronics integrated in the device for the measurement of at least one parameter, and
(b) measuring the auxiliary variable Ω, also referred to as the ratio of the modulation depths, and
(c) positioning a shield between emitter and receiver or selecting a sufficiently long light path length such that auxiliary variable Ω reaches a minimum of 0.5 or less for the following set of conditions:
at an oxygen saturation level in the arterial (pulsating) human blood of 99-100%
for a wavelength combination of 730 nm and 880 nm specified as center wavelengths of typical symmetrical LED spectra
or a computable corresponding omega minimum for other conditional terms.

13. A method for monitoring tissue optical values such as absorption, analyzing optic plethysmographic signals such as determining the pulse rate or mainly of the arterial oxygen saturation in the blood, in particular for the realization of pulse oximetry in the auditory canal, comprising:
(a) introducing into an auditory canal a device comprising a sensor-carrier (2) adapted to being placed in the auditory canal, at least one sensor component (1) for measuring at least one parameter when placed in the auditory canal, said sensor positioned on and/or connected with which sensor carrier, a sensor-carrier-positioning-element (3) connected with or merging into the sensor-carrier (2), and electronics integrated in the device for the measurement of at least one parameter, and
(b) measuring the auxiliary variable Ω, also referred to as the ratio of the modulation depths, and
(c) positioning a shield between emitter and receiver or selecting a sufficiently long light path length such that auxiliary variable Ω reaches a minimum of 0.5 or less for the following set of conditions:
Circummission Light Path
Circummission Pulse Oximetry
or a computable corresponding omega minimum for other conditional terms.

14. The method according to claim 12, wherein the parameter is selected from the group comprising the body temperature, the oxygen saturation of the blood especially the arterial blood, the heart rate, electric parameters of the heart (ECG), the respiration rate, the blood pressure, the concentration of substances dissolved in the blood, the concentration of substances present in the tissue, the physical activity, mechanical parameters of the body and parameters in relation to sleep and vigilance.

15. The method according to claim 12, wherein the device comprises at least one sensor-carrier (2) with a sensor component (1) in form of at least one light emitter (15) and of at least one light receiver (16), and wherein the device provides means for preventing that light travels directly from the light emitter (15) to the light receiver (16) without transilluminating the tissue i.e. means for avoiding shunt light.

16. The method according to claim 12, wherein the light path through the tissue of the auditory canal (12) follows the principles of is a Circummission Pulse Oximetry.

17. The method according to claim 15, wherein the means is a clamp shaped, disc-shaped or umbrella-shaped element in which the light emitter (15) is positioned on one side and the light sensor (16) on the other side, or an umbrella-shaped double sensor-carrier (17) as to which the light emitter (15) is positioned on one umbrella and the light sensor (16) in the other umbrella.

18. The method according to claim 12, wherein for the purpose of the maximization of the light path length or to the suppression of shortened light paths, means is applied of heavily light-diminishing mainly light-blocking cover of more than half of the auditory canal's half-circumference in the area of the net light path (18) to both sides of the light sensor (16), mainly in form of an arched, umbrella-shaped sensor-carrier (2) or of several such sensor-carriers (17).
